(19) United States
(12) Patent Application Publication
(10) Pub. No.: US 2013/0183686 A1
(43) Pub. Date: Jul. 18, 2013

(54) METHOD OF EVALUATING IMMUNOSUPPRESSION

(71) Applicants: Oleh G. Pankewycz, Amherst, NY (US); Mark R. Laftavi, Buffalo, NY (US); Hans Minderman, Lockport, NY (US)

(72) Inventors: Oleh G. Pankewycz, Amherst, NY (US); Mark R. Laftavi, Buffalo, NY (US); Hans Minderman, Lockport, NY (US)

(73) Assignee: Donna Davidovich

(21) Appl. No.: 13/664,949

(22) Filed: Oct. 31, 2012

Related U.S. Application Data
(63) Continuation-in-part of application No. 13/128,292, filed on Sep. 1, 2011.
(60) Provisional application No. 61/553,324, filed on Oct. 31, 2011.
(60) Provisional application No. 61/113,381, filed on Nov. 11, 2008.

(51) Int. Cl. G01N 33/68

(52) U.S. Cl. CPC G01N 33/6872
USPC 435/7.24; 435/7.1

(57) ABSTRACT

Provided is a method for determining immunosuppression in an individual. The method entails testing blood cells for nuclear NFkB and/or nuclear NFAT. The blood cells can be from a sample of blood from an individual. The cells can be contacted with an activating agent to obtain activated cells, and the amount of nuclear NFkB and/or NFAT can be compared to a control. An amount of nuclear NFkB and/or NFAT that is higher than the control is considered to be indicative of insufficient immunosuppression in the individual. An amount of nuclear NFkB and/or NFAT that is lower than the control is considered to be indicative of excessive immunosuppression in the individual. An amount of nuclear NFkB and/or NFAT that is the same as the control is considered to be indicative of an appropriate amount of immunosuppression in the individual.

### CROSS REFERENCE TO RELATED APPLICATIONS

This application claims priority to U.S. Provisional Patent Application No. 61/553,324, filed on Oct. 31, 2011, and is a continuation in part application of U.S. application Ser. No. 13/128,292, filed Sep. 1, 2011, which is a U.S. national phase application of international application no. PCT/US09/64010, filed on Nov. 11, 2009, which in turn claims priority to U.S. Provisional Patent Application No. 61/113,381, filed on Nov. 11, 2008, the disclosures of each of which are hereby incorporated by reference.

### STATEMENT REGARDING FEDERALLY SPONSORED RESEARCH

This invention was made with government support under contract no. 1R21CA126667 awarded by the National Institutes of Health. The government has certain rights in the invention.

### FIELD OF THE INVENTION

The present invention relates generally to determining immunosuppression, and more particularly to determining immunosuppression in an individual by measuring nuclear NFκB and/or Nuclear Factor of Activated T-cells (NFAT) in blood cells obtained from the individual.

### BACKGROUND OF THE INVENTION

Patients who receive a solid organ transplant must take immunosuppressive therapy to prevent rejection. Contemporary immunosuppressive protocols call for continuous therapy for the life-span of the transplanted organ. Potentially life-long anti-rejection therapy has many adverse consequences including increased rates of infections and cancers, worsening cardiovascular risk factors and bone disease. Therefore, individualized or minimized immunosuppression is a major clinical goal, saving the highest levels of immunosuppressive therapy for those patients at highest risk of rejection and graft loss. Currently, there is an ongoing need for a reliable non-invasive test that allows for such patient directed immunosuppressive therapy. The present invention meets this need.

### SUMMARY OF THE INVENTION

The present invention provides a method for determining immunosuppression in an individual. The invention can be used to determine immunosuppression in any individual undergoing any type of immunosuppression with any immunosuppressive agent. Immunosuppression is considered to be a measure of immune competence.

The method comprises obtaining a sample of blood from an individual, contacting cells in the blood sample with an activating agent to obtain activated cells, and processing the cells to measure nuclear NFκB and/or NFAT in the activated cells. The amount of nuclear NFκB and/or NFAT in the activated cells is informative with respect to immunosuppression. The amount of NFκB and/or NFAT can be compared to a suitable control. Generally, the nuclear NFκB and/or NFAT is determined in T cells, such as CD4+ T cells, CD8+ T cells, and combinations thereof.

In another embodiment, the invention provides a method for determining nuclear translocation of NFkB and/or NFAT for an individual undergoing immunosuppression. The method comprises obtaining a biological sample comprising nucleated blood cells from the individual, contacting the cells in the sample with an activating agent to obtain activated cells, and testing the cells to determine an amount of nuclear NFκB and/or NFAT in the activated cells.

In another embodiment, the method includes determining nuclear NFkB and/or NFAT in nucleated blood cells that are naïve to an immunosuppressive agent. In certain embodiments this is performed using image flow cytometery to determine a ratio of nuclear to cytoplasmic NFkB and/or NFAT. A comparison of the amount of nuclear NFkB and/or NFAT to a suitable control, or to the amount of cytoplasmic NFkB or NFAT, respectively, can be used to establish a baseline amount of nuclear NFkB and/or NFAT, as the case may be. The baseline levels of nuclear amounts of these markers are useful for example, for predicting clinical presentation for individuals who are being treated with immunosuppressant agents, or who are being considered for immunosuppressant therapy. Subsequent to baseline determination, nucleated blood cells from the patient can be tested alone to ascertain nuclear translocation potential of NFkB and/or NFAT. In certain embodiments, nucleated blood cells from a patient who is a candidate for a transplantation can be mixed with nucleated blood cells from a candidate donor, and such mixtures can be tested for translocation potential of NFkB and/or NFAT in response to any of a variety of immunosuppressant agents and combinations thereof, using any of a variety of activating agents.

Either or both of nuclear NFκB and NFAT can be used for determining immunosuppression generally, and for any immunosuppressive agent. In certain embodiments, the degree of nuclear NFκB is informative for immunosuppressants which are non-calcineurin inhibitors, such as mTOR inhibitors (i.e., rapamycin), while the degree of nuclear NFAT is informative for calcineurin inhibitors (i.e., tacrolimus and cyclosporine).

In general, an amount of nuclear NFκB and/or NFAT that is higher than a control is considered to be indicative of insufficient immunosuppression in the individual. An amount of nuclear NFκB and/or NFAT that is lower than a control is considered to be indicative of excessive immunosuppression in the individual. An amount of nuclear NFκB and/or NFAT that is the same as a control is considered to be indicative of an appropriate amount of immunosuppression in the individual.

In one embodiment, the method comprises obtaining a second whole blood sample from the individual (or dividing a first sample into first and second samples), wherein cells in the second sample are not activated. The amount of nuclear NFκB and/or NFAT in the non-activated cells can be used for comparison to the amount of nuclear NFκBa and/or NFAT in the activated cells to establish a baseline amount of nuclear NFκB and/or NFAT prior to activation for use as a control.

The blood sample obtained from the individual comprises immune cells that include but are not limited to T cells, monocytes, polymorphonuclear leukocytes, eosinophils, and combinations thereof. In one embodiment, the cells analyzed in the method of the invention comprise CD3+ cells, CD4+ cells, CD8+, CD20+ cells, or a combination thereof. Thus, the blood sample contains lymphocytes that can be phenotyped according to particular surface antigens and analyzed in the method of the invention.

In one embodiment, the method further comprises communicating to a health care provider a determination that an amount of nuclear NFκB and/or NFAT is indicative of insufficient, excessive or appropriate immunosuppression in the individual.

In one embodiment, the method further comprises modifying immunosuppression dosing for the individual subsequent to determining insufficient or excessive immunosuppression.

### BRIEF DESCRIPTION OF THE DRAWINGS

FIG. 1 provides a graphical representation of a comparison of imaging flow cytometry data with data obtained from a commercially available immunosuppression test. Discordant data were observed for 2/9 patients in which cases the patients suffered acute rejection (*) or viral infections (**), and thus demonstrates superiority of the present invention in predicting clinical outcomes.

FIG. 2 provides a graphical representation of a comparison of immune response of transplant recipients receiving immunesuppressive therapy (n=5) and healthy donors (n=4). Immune response was measured as the amount of nuclear NFκB translocation (similarity score) in CD3, CD4 and CD8 T-cells in response to ex-vivo stimulation to PMA/ion or TNFα. Bars represent mean values with standard deviations.

FIG. 3 provides a graphical representation of a correlation between NFkB activation potential and clinical presentation in CD4+ cells of patients receiving tacrolimus.

FIG. 4 provides a graphical representation of a correlation between NFkB activation potential and clinical presentation in CD4+ cells of patients receiving rapamycin.

FIG. 5 provides a graphical representation of a correlation tacrolimus inhibition of PMA/Ionomycin-induced NFAT activation in healthy donor CD4+ cells in culture medium.

FIG. 6 provides a graphical representation of a correlation Tacrolimus inhibition of PMA/Ionomycin-induced NFAT and NFkB activation in healthy donor CD4+ cells in plasma.

FIG. 7 provides a graphical representation of a correlation Tacrolimus plasma pharmacokinetics of 16 cases after first oral dose.

FIGS. 8 and 9 provide a graphical representations of the correlation of PMA/Ionomycin-induced NFAT1 translocation in Jurkat cells incubated in plasma obtained between 1 h and 12 h following in vivo tacrolimus administration.

FIG. 10 provides a graphical representation of a correlation NFAT1 baseline activity in transplant recipients receiving tacrolimus-based versus rapamycin-based immunosuppression.

### DETAILED DESCRIPTION OF THE INVENTION

The present invention provides a method for determining immunosuppression in an individual. The method is based on our discovery that quantification of nuclear NFκB and NFAT in immune cells is meaningful and can be used in various embodiments to determine the state of immunosuppression in the individual. The method is applicable to any therapeutic intervention that directly or indirectly targets the activity of the NFkB and/or NFAT signaling pathways, and includes but is not necessarily limited to therapies that relate to the field of transplant medicine, autoimmune diseases and cancer, including but not limited to blood cancer.

The method involves measuring in blood cells an amount of nuclear NFkB and/or nuclear NFAT. In one embodiment, the invention involves determining the amount of one or both of these markers that translocates to the nucleus after the cells have been activated by exposure to an activating agent. Thus, in one embodiment, the invention entails determining a change in nuclear NFκB and/or NFAT that occurs in response to activation of certain blood cells obtained from the individual. In an alternative embodiment, a ratio of cytoplasmic to nuclear NFκB and/or NFAT can be determined in the activated cells.

“Immunosuppression” as used herein refers to the effect on the immune system of an individual elicited by administration of one or more immunosuppressive agents to the individual Immunosuppression is considered a type of immune competence, meaning the degree to which an individual can mount an appropriate immune response against a foreign antigen Immune competence of particular types of immune cells can also be determined in performance of the method of the invention. As noted above, the invention includes determining immunosuppression, as well as the effect of any agent that directly or indirectly targets the activity of the NFkB and/or NFAT signaling pathways.

In connection with immunosuppression, a wide variety of immunosuppressive agents are known in the art and are routinely administered to individuals for a variety of purposes. The invention is suitable for determining immunosuppression in any individual undergoing any type of immunosuppression with any immunosuppressive agent, which include but are not limited to calcineurin inhibitors (CNI) (i.e., tacrolimus or cyclosporin), mycophenolic acid (MPA) (i.e., Cellcept or Myfortic), sirolimus (i.e., Rapamune or Certican) and prednisone. Thus, it is considered that the method is suitable for evaluating the immunosuppression status of any mammal, including male and female humans, and ranging in age from infants to the elderly. In certain aspects of the invention, nuclear NFκB is informative for assessing immunosuppression for individuals who are undergoing or are being considered for therapy with non-calcineurin inhibitors, while nuclear NFAT is informative for assessing immunosuppression for individuals who are undergoing or are being considered for therapy calcineurin inhibitors.

In one embodiment, the invention is used to evaluate immunosuppression in an individual who has a transplanted organ or other tissue and is undergoing immunosuppression therapy to reduce the likelihood that the transplant will be rejected. In various embodiments, the individual has a transplanted kidney, pancreas, heart, lung, hand, face, skin, bone, bone marrow, cartilage, ligament or muscle. The transplant may also be a xenographic transplant, or a transplantation of a synthetic substance.

“Activation” or “activating” or to “activate” cells are terms understood to those skilled in the art. In general, activation comprises causing certain immune cells described more fully below to undergo an alteration in gene expression such that the cells can participate in effecting a more vigorous immune response relative to non-activated cells. Evidence of activation includes but is not limited to translocation of NFκB and/or NFAT from the cytoplasm to the nucleus, as well as production of various cytokines by activated cells which promote, among other well known effects, an inflammatory response.

The method of the invention in various embodiments comprises obtaining a sample of blood from an individual, contacting cells in the blood sample with an activating agent to obtain activated cells, measuring nuclear NFκB and/or NFAT in the activated cells, and comparing amount of nuclear NFκB and/or NFAT in the activated cells to a control, suitable controls being more fully described below. The amount of NFκB and/or NFAT that is translocated to the nucleus is considered to be a measure of NFκB and/or NFAT translocation potential, respectively. It is considered that the NFκB and/or NFAT translocation potential is indicative of the immune response that the cells would exhibit upon encountering a foreign antigen, such as an antigen displayed by transplanted tissue.

Determining more nuclear NFκB and/or NFAT than in the control by performing the method of the invention is considered to be indicative of insufficient immunosuppression in the individual. In one embodiment, insufficient immunosuppression is exemplified by an individual who is undergoing immunosuppression therapy and experiences rejection of a transplanted organ or tissue. Those skilled in the art are familiar with clinical criteria used to determine whether any particular transplanted organ or tissue is being rejected. Less nuclear NFκB and/or NFAT relative to the control is considered to be indicative of excessive immunosuppression in the individual. In one embodiment, excessive immunosupression is exemplified by an individual who is undergoing immunosuppression therapy and experiences viral infections more frequently and/or with more severe symptoms than expected had the individual been receiving an appropriate amount of immunosuppression. Those skilled in the art are familiar with criteria used to determine whether any particular individual who is undergoing immunosuppression therapy is experiencing viral infections more frequently and/or with more severe symptoms than would be expected if an appropriate amount of immunosuppression was being provided to the individual. An amount of nuclear NFκB and/or NFAT that is the same as a control is considered to be indicative of an appropriate amount of immunosuppression in the individual. One non-limiting example of an individual who has an appropriate amount of immunosuppression is an individual who has a transplanted organ or other transplanted tissue, is receiving immunosuppression, and is not rejecting the transplanted organ or other tissue, and is not experiencing viral infections.

In certain embodiments, determining that an individual has insufficient immunosuppression via assaying the amount nuclear NFκB can result in changing the treatment regime of the individual, such as by increasing the dosage of the immunosuppresant, or by adding a non-calcinuerin inhibitor, or replacing a calcinuerin inhibitor with a non-calcinuerin inhibitor. Likewise, in certain embodiments, determining that an individual has excessive immunosuppression via assaying the amount nuclear NFκB can result in changing the treatment regime of the individual, such as by decreasing the dosage of the immunosuppresant, or by adding a non-calcinuerin inhibitor, or replacing a calcinuerin inhibitor with a non-calcinuerin inhibitor.

In certain embodiments, determining that an individual has insufficient immunosuppression via assaying the amount nuclear NFAT can result in changing the treatment regime of the individual, such as by increasing the dosage of the immunosuppresant, or by adding a calcinuerin inhibitor, or replacing a non-calcinuerin inhibitor with a calcinuerin inhibitor. Similarly, in certain embodiments, determining that an individual has excessive immunosuppression via assaying the amount nuclear NFAT can result in changing the treatment regime of the individual, such as by decreasing the dosage of the immunosuppresant, or by adding a calcinuerin inhibitor, or replacing a non-calcinuerin inhibitor with a calcinuerin inhibitor.

The method of the invention is demonstrated to be superior to the commercially available assay marketed under the trade name ImmunKnow (Cylex, Columbia, Md.) assay in predicting clinical outcome for immunosuppressed individuals. In particular, our results demonstrate that in one individual, the ImmuKnow assay indicated heightened immunity yet the patient suffered from above normal viral infections. However, analysis of blood cells from that individual using an embodiment of the present invention showed a markedly diminished ability to translocate NFkB, which is consistent with excessive immunosuppression. In another individual, the ImmuKnow assay predicted excessive immunosuppression, but the patient had acute rejection. Analysis of blood cells from that individual using an embodiment of the present invention showed a greater than normal ability to translocate NFκB from cytoplasm to the nucleus, which is consistent with inadequate immunosuppression, and thus correctly reflected the actual clinical outcome for this patient.

As will be recognized from the foregoing, the present invention involves detecting amounts of nuclear NFκB in stimulated immune cells. NFκB is a ubiquitously expressed transcription factor that regulates many normal cellular processes. NFκB transcription proteins include a collection of proteins that exist as dimers of two classes of proteins. The Class A proteins, p105 and p100, do not ordinarily act as transcription factors unless they undergo limited proteolysis to the shorter proteins p50 and p52 respectively. Activated Class A proteins bind to Class B proteins c-Rel, RelB and p65 to form the activated heterodimeric transcription complex. The p65 subunit of NFκB is also referred to in the art as RelA, Rel A and RELA. The most avid dimer and the major NFκB complex is p50/RelA. The activation of NFκB is usually transient with nuclear localization lasting 30 to 60 minutes followed by rapid egress of NFκB back to the cytoplasm. Thus, the presence of nuclear NFκB is considered to represent a recent activation event.

It will be recognized by those skilled in the art that any subunit of which nuclear NFκB is comprised can be detected during performance of the method of the invention. For example, detecting any homo- or heterodimeric complexes containing NFκB p65 (RELA/p65), RELB, NFκB /p105, NFκB1/p50, REL and NFκB2/p52 can be performed to quantify nuclear NFκB. In one embodiment, nuclear NFκB is determined by detecting the p65 subunit of NFκB. In connection with this, any particular individual may have polymorphisms and/or other allelic variation in p65 (as well as other NFκB subunits), but it is considered that all such potential variations can be detected using commercially available reagents. For example, it is considered that p65 expressed in blood cells obtained from any individual human can be detected using any of a variety of commercially available anti-p65 antibodies, such as those available from ABCAM (Cambridge, Mass., USA) and a variety of other commercial vendors. In one embodiment, nuclear NFκB is determined by detecting human p65 protein that has the amino acid sequence designated by GenBank accession number CAA80524.2, Nov. 14, 2006 entry, which is incorporated herein by reference.

NFAT activity is regulated by its calcineurin-dependent dephosphorylation which allows NFAT to translocate from the cytoplasm, where it resides in its phosphorylated inactive state, to the nucleus, where dephosphorylated NFAT can bind to the promoter sites of its target genes. Calcineurin is a direct target of tacrolimus and cyclosporine, two important immunosuppressive drugs, thus inhibition of the nuclear translocation potential of NFAT is a pharmacodynamic measurement for the activity of tacrolimus and cyclosporine. There are five members in the NFAT transcription factor family. NFAT5 responds to osmotic stress while NFAT3 is primarily found in non-T-cells. NFAT4 is primarily found in thymocytes. NFAT 1 and 2 have significant functional redundancy. The Examples presented herein studied NFAT1, but any member of the NFAT transcription factor family could be determined in performing the method of the invention. The human NFAT sequences associated with the following GenBank accession numbers are incorporated into the application as they are described in GenBank as of the effective filing date of the present disclosure: human NFAT1(NM_{—}012340); NFAT2 (NM_{—}172390); NFAT3(NM_{—}001136022); NFAT4 (NM_{—}173165); NFAT5 (NM_{—}138714).

The blood sample obtained from the individual is one that comprises cells that are suitable for analysis using the method of the invention. Such cells can include any nucleated blood cells, and include but are not particularly limited to lymphocytes and other cells that participate in cell mediated and/or humoral immune responses. For example, cells that are present in the blood sample obtained from the individual and that can be analyzed in the method of the invention include but are not necessarily limited to T cells, monocytes, polymorphonuclear leukocytes, eosinophils, B cells, and combinations thereof. Those skilled in the art are familiar with known markers and methods that can be used to detect and differentiate these cell types from one another. For instance, T cells are CD3+ cells that can be further distinguished from each other by subtype markers, such as CD4+ (T helper cells) and CD8+ (cytotoxic T cells). Additionally CD19+ and/or CD20+ cells (B cells) and CD16+ cells (natural killer cells) can be analyzed in performing the method of the invention.

When cells in the sample of blood are activated, the activating agent is not particularly limited, and a wide variety of suitable activating agents are known in the art and are commercially available. Some non-limiting examples of activating agents suitable for use in the present invention include phytohemagglutinin (PHA), phorbol 12-myristate 13-acetate (PMA) with ionomycin (ion), tumor necrosis factor alpha (TNF-alpha), and anti-CD3/CD28 antibodies.

Suitable controls for use in the method of the present invention include but are not limited to a standardized curve, cell lines with known proportions of cytoplasmic NFκB that translocates from the cytoplasm to the nucleus upon activation, or any other standardized parameter(s) that delineates a ratio of nuclear NFκB to cytoplasmic NFκB in blood cells after activation and that indicates appropriate, excessive or insufficient immunosuppression. Those skilled in the art will recognize how to interpret a comparison of the amount of nuclear NFκB to any particular control. For example, an amount of nuclear NFκB that is within the range of the amount of NFκB determined from lymphocytes obtained and activated from stable transplant patients (e.g., those patients not undergoing rejection or experiencing above normal viral infections) is considered to be the same as the control (e.g., the same as a normal control) and can be recognized as such by those skilled in the art. Likewise, an amount of nuclear NFκB that is above the range of the amount of nuclear NFκB for a normal control can be readily recognized, as can an amount of nuclear NFκB that is below a range of the amount of nuclear NFκB for the normal control.

In one embodiment, the blood sample obtained from the individual is divided into an experimental and a second blood sample. Cells in the experimental sample are contacted with the activating agent; cells in the second whole blood sample are not contacted with the activating agent (i.e., the second whole blood sample comprises non-activated cells). The amount of nuclear NFκB in the second whole blood sample can be determined according to the method of the invention and is considered to be a non-activated amount of nuclear NFκB. Thus, the non-activated cells can be used to establish a baseline, or non-activated, amount of nuclear NFκB for comparison with the amount of nuclear NFκB in the activated cells.

As described above, the activating agent used to activate the cells is not particularly limited. Those skilled in the art are also familiar with the incubation parameters used to activate any particular cell type(s) using any particular activating agent. In one embodiment, the activating agent is added to a blood sample and the cells and activating agent added thereto are incubated together for a period of from 1 minute to 60 minutes, including all integers there between. In one embodiment, the incubation period is 30 minutes. In one embodiment, the incubation period is not more than from 1 minute to 60 minutes, including all integers there between.

In one embodiment, the blood sample obtained from the individual is a sample of whole blood. It is an unexpected advantage of the present invention that cells in the whole blood, such as CD3+, CD4+, CD8+ cells, can be contacted with the activating agent in the sample of whole blood. This is considered to be a more accurate representation of the normal in vivo environment of the cells, relative to first separating the CD3+, CD4+, CD8+ cells out of whole blood (i.e., by using ficol gradients to isolate peripheral blood cells (PBL)) and then contacting the separated cells with the activating agent. Moreover, use of whole blood permits the assay to be completed in a much shorter amount of time than if separated immune cells are used. For example, the entire assay can be completed in not more than from 2 to 4 hours, as opposed to much longer periods for assays that rely on separated cells.

In one embodiment, after contacting the cells with the activating agent, cells are immunophenotyped with commercially available fluorescently labeled antibodies, after which the red blood cells are removed from the whole blood sample. Red blood cells can be removed using conventional techniques, such as by lysing using a hyptonic solution under conventional conditions which does not also result in lysis of the activated cells. In one embodiment, a commercially available lyse/fix solution (Becton Dickenson) can be used.

In one embodiment, prior to determining nuclear NFκB, the cells in which the nuclear NFKB is to be determined are incubated with an antibody specific to a relevant NFKB subunit, wherein the antibody is conjugated to a fluorescent marker. In a similar embodiment, prior to determining nuclear NFAT, the cells in which nuclear NFAT is to be determined are incubated with an antibody specific to a relevant NFAT, wherein the antibody is conjugated to a fluorescent marker. In various embodiments, nuclear NFκB and NFAT can be determined concurrently, or sequentially, and for the same cell population, or for separate cell populations. For certain applications, the antibody directed to a NFκB subunit can be conjugated to with a different fluorescent marker than the antibody directed to NFAT so that they can be separately identified and measured.

In more detail, in various embodiments of the invention, the relative amounts of nuclear NFκB and/or nuclear NFAT can be determined in activated and/or non-activated samples by analysis with commercially available devices and/or systems that can differentiate and quantify the nuclear and cytoplasmic NFκB and/or NFAT, such as by a variety of digital microcopy-based imaging techniques. For example, activated and/or non-activated preparations of cells could be fixed and analyzed using detectably labeled antibodies to NFκB (such as to p65) and/or NFAT, as applicable, and known reagents to stain or otherwise identify the nucleus such that the nuclear (and if desired cytoplasmic) NFκB, and/or NFAT, as applicable, can be distinguished from one another. Suitable nuclear stains include but are not limited to 4′,6-diamidino-2-phenylindole (DAPI), Hoechst stains, Haematoxylin, Safranin, Carmine alum, and DRAQ5.

In one embodiment, nuclear NFKB and/or nuclear NFAT can be determined using imaging flow cytometry. For example, the amount of nuclear NFKB in activated and non-activated cells can be determined for CD3+, CD4+, CD8+, and/or CD20+ cells using detectably labeled antibodies directed to the CD3+, CD4+, CD8+ and/or CD20+ molecules, as well as detectably labeled antibodies to NFκB. Similarly, the amount of nuclear NFAT in activated and non-activated cells can be determined for CD3+, CD4+, CD8+, and/or CD20+ cells using detectably labeled antibodies directed to the CD3+, CD4+, CD8+ and/or CD20+ molecules, as well as detectably labeled antibodies to NFAT. The nuclei of the cells can be simultaneously visualized using a suitable nuclear stain that can be detected by an imaging flow cytometer. In one embodiment, the nuclear stain is DAPI. In another embodiment, the nuclear stain is DRAQ5.

In certain aspects of the invention, determining nuclear NFκB and/or nuclear NFAT is performed using an imaging flow cytometer. In one embodiment, the imaging flow cytometer is an imaging flow cytometer device described in U.S. Pat. No. 7,522,758, the disclosure of which is hereby incorporated by reference. Such image flow cytometers are able to quantitatively measure the relative proportion of NFκB and/or NFAT that is located in the cytoplasm versus the amount in the nucleus. This proportion allows for measuring, among other parameters, the relative activation state of a given cell population between two samples obtained from two different patients or the same patient at different times obtained under differing clinical circumstances. For example, by dual staining for p65 or NFAT1 and cell surface markers such as CD4, CD8, CD19/20 and CD16, the image flow cytometer can measure the relative NFkB and NFAT activation in T helper cells, T cytotoxic cells, B cells and Natural Killer cells, respectively. The ability to assess activation states of multiple different cell types represents a major advance compared to the CD4 limited ImmuKnow assay. Thus, in one embodiment, the invention provides for determining a composite result obtained from all or a subset of cell types. The resting state compared to the activated state of peripheral blood lymphocytes (PBL) may accurately reflect in vivo events such as infection and rejection.

In one embodiment, a relative amount of NFκB and/or NFAT present in the nucleus can be represented by a similarity score determined using an imaging flow cytometer system such as that described in U.S. Pat. No. 7,522,758. In general, the smaller the similarity score, the less nuclear translocation of NFκB and/or NFAT, and vice versa. More specifically, the similarity score is considered to be a log transformed Pearson's Correlation coefficient of the pixel by pixel intensity correlation between the NFκB and/or NFAT and nuclear stained (i.e., DRAQ5 image) which is calculated as a quantifiable parameter for the degree of NFκB and/or NFAT translocation to the nucleus. The similarity score (+ or −) is determined from the slope of the regression line while it takes its value from how well the individual pixel data points fit the regression line (Pearson correlation). A very low degree of nuclear translocation yields a highly negative similarity score while a very high degree of nuclear translocation yields a highly positive similarity score. It will therefore be recognized that, in one embodiment a low degree of nuclear translocation can have anti-similar p65 and DRAQ5 images, while similar p65 and DRAQ5 images can yield a positive similarity score. Thus, in one embodiment, following stimulation, a negative similarity score obtained using an imaging flow cytometer system is indicative of excessive immune suppression, while a highly positive similarity score obtained using an imaging flow cytometer system is indicative of insufficient immune suppression. A standardized similarity score or ranges of similarity scores can accordingly be used as a control when performing the method of the invention.

In various embodiments, the invention provides one or more isolated population of immune cells. In certain aspects the isolated populations of cells are present in an imaging flow cytometer system. In certain embodiments, the invention provides a plurality of distinct isolated immune cell populations, wherein a first immune cell population comprises or consists of immune cells characterized by nuclear NFκB complexed with a detectably labeled antibody that is specific for NFκB, and a second immune cell population which comprises or consists of immune cells characterized by nuclear NFAT complexed with a detectably labeled antibody that is specific for NFAT. The first and/or second cell populations can comprise or consist of certain types of immune cells, such as CD3+ cells, CD4+ cells, CD8+, and CD20+ cells. The isolated cell populations can be obtained from the same individual, or can comprise cells obtained from different individuals, such as an organ donor and an intended or actual transplant recipient. The isolated cell populations can be activated, or non-activated and thus are useful for determining baseline and activated nuclear NFκB and/or NFAT.

Determining an amount of nuclear NFκB and/or NFAT in activated cells from an individual, wherein the amount is different from a control, is considered to be indicative that the individual is a candidate for an alteration of his or her immunosuppression therapy. For example, an individual for whom performing the method of the invention indicates insufficient immunosuppression could be recommended for an increase in dosing, or for a change to a different immunosuppression agent. Likewise, an individual for whom performing the method of the invention indicates excessive immunosuppression could be recommended for a decrease in dosing, or for a change to a different immunosuppression agent. An individual for whom performing the method of the invention indicates an appropriate amount of immunosuppression could be recommended for no change in immunosuppression regime.

In one embodiment the method includes determining nuclear NFkB and/or NFAT in nucleated blood cells that are naïve to an immunosuppressive agent. Such cells can be obtained, for example, for an individual who has not previously been treated with an immunosupprsive agent. The invention includes comparing the amount of nuclear NFkB and/or NFAT to a suitable control, or to the amount of cytoplasmic NFkB or NFAT, respectively, to establish a baseline amount of nuclear NFkB and/or NFAT. The baseline levels of nuclear amounts of these markers are useful for example, for generating an immunosuppressive treatment protocol for individuals who are being treated with immunosuppressant agents, or who are being considered for immunosuppressant therapy. Subsequent to baseline determination, nucleated blood cells from the patient can be tested alone or in combination with other cells to ascertain nuclear translocation potential of NFkB and/or NFAT. In particular embodiments, nucleated blood cells from a patient who is a candidate for a transplantation can be mixed with nucleated blood cells from a candidate donor. These cell mixtures can be tested for translocation potential of NFkB and/or NFAT in response to any of a variety of immunosuppressant agents and combinations thereof, using any of a variety of activating agents, to further refine a treatment regime for any given individual who is a candidate for immunosuppressive therapy.

The method of the invention can be repeated to monitor the immunosuppression status of an individual over time. For example, the invention can be used to evaluate whether modifications of the immunosuppression therapy of an individual should be considered and/or implemented. The method of the invention can also be performed prior to initiation of immunosuppression therapy and compared to a sample(s) of blood obtained from the individual after initiation of immunosuppression therapy to evaluate the efficacy of the therapy.

In one embodiment, the method of the invention comprises communicating to a health care provider the result of determining an amount of nuclear NFκB and/or NFAT in activated cells from an individual that is different from, or the same as, a control. The health care provider can be any individual who participates in making health care decisions for the individual. In another embodiment, the invention comprises communicating to an insurance provider the result of determining an amount of nuclear NFκB and/or NFAT in activated cells from an individual that is different from, or the same as, a control.

In one embodiment, the method of the invention further comprises recommending an alteration of an immunosuppression therapy subsequent to determining an amount of nuclear NFκB and/or NFAT that is different from a control, such as a control comprised of the amount of nuclear NFκB and/or NFAT observed in activated cells obtained from stable transplant recipients. This embodiment may further comprise altering the immunosuppression therapy for the individual.

In one embodiment, the method comprises fixing the result of determining the amount of nuclear NFκB and/or NFAT in a tangible medium of expression, such as a digital medium, including but not limited to a compact disk, DVD, or any other portable memory device. Thus, the invention also provides a device or other tangible medium that contains a machine or human readable result from determining nuclear NFκB and/or NFAT that is different from that observed in a control.

The following Examples are intended to illustrate but not limit the invention.

### EXAMPLE 1

We measured the degree to which NFκB translocation in peripheral T cells was impaired by immune-suppressive therapy using a commercially available imaging cell flow cytometer (Amnis Corporation, Seattle, Wash.).

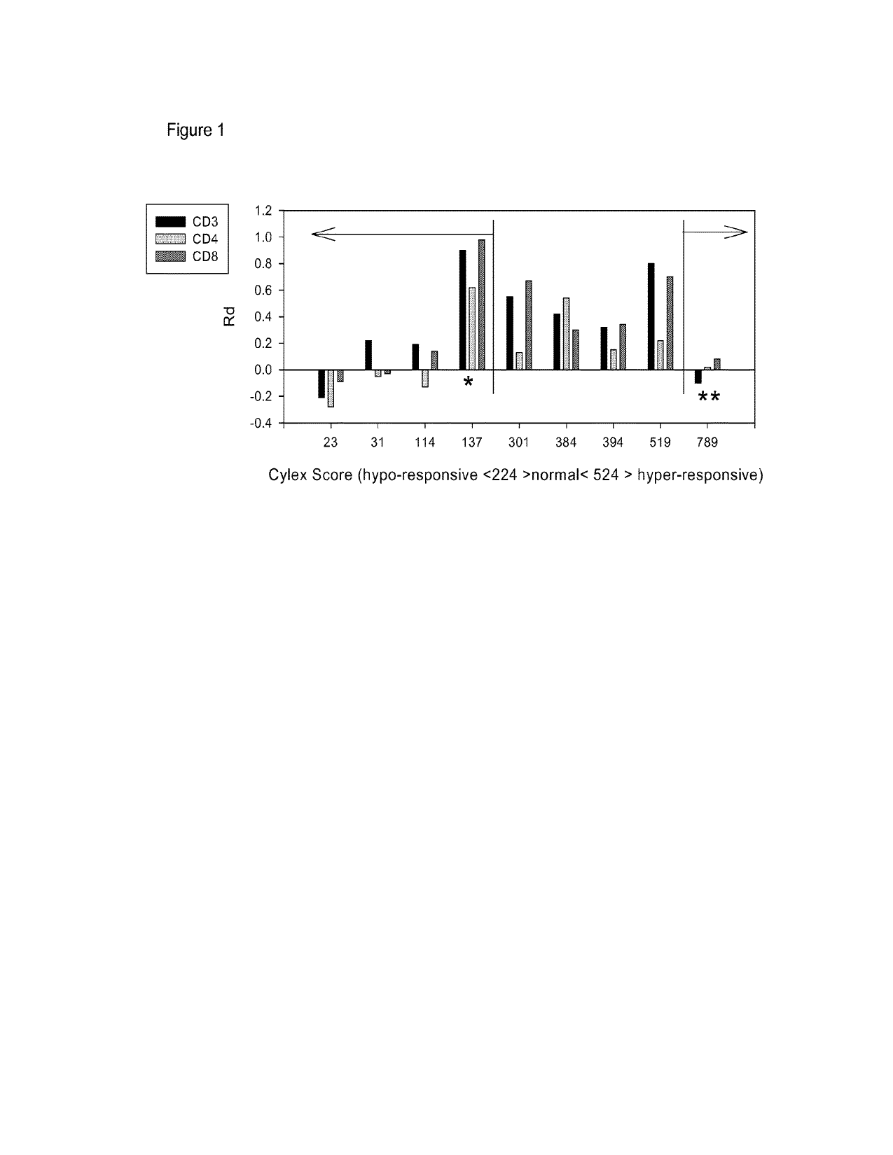

Peripheral blood cells from 9 transplant recipients were isolated, stimulated in culture with PMA/ionomycin (30 min), stained for T cell surface markers and NFκB (p65) and the relative amount of nuclear NFκB in resting and activated CD3, CD4 and CD8 positive T-cell subsets was compared. Results were then correlated with clinical response (stable graft function, infections and rejections) and ImmuKnow assay results. The assay correlated well with results obtained in parallel using the commercially available ImmuKnow product according to manufacturer's instructions, (FIG. 1) but there were 2 major discrepancies. In one patient (** in FIG. 1), the ImmuKnow assay levels indicated heightened immunity yet the patient suffered from major viral infections. In this patient, the imaging cell flow cytometry correctly showed a markedly diminished ability to translocate NFκB consistent with over-immunosuppression. In the other case (* in FIG. 1), the ImmuKnow assay levels predicted an excessive level of immunosuppression, but the patient had acute rejection. The imaging cell flow cytometry assay showed a greater than normal ability to translocate NFκB consistent with inadequate immunosuppression, thus correctly reflecting the clinical outcome. Thus, the present invention provides improved assessment of the degree of immunosuppression in an individual and is expected to more accurately predict clinical outcome across a broad range of patients.

### EXAMPLE 2

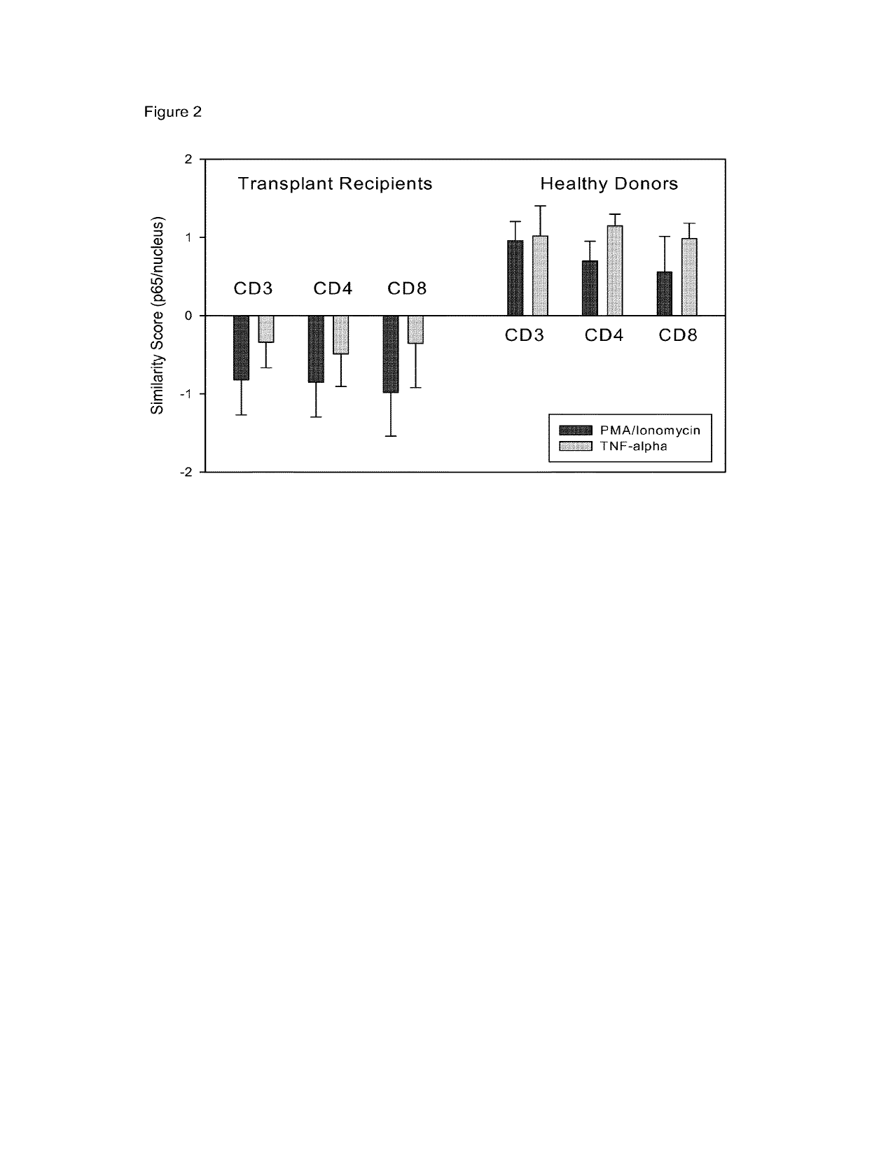

The assay described in Example 1 was modified to perform the stimulation and cell surface labeling in whole blood to enable the method to be performed in the normal environment of the cells and to permit faster performance of the assay. Using this approach, the immune response of 5 transplant patients as compared to 4 normal donors to stimulation to TNFα or PMA/ion was compared. The data depicted in FIG. 2 demonstrate that using a similarity score read-out for nuclear NFκB as a measure for immune response, a striking difference could be observed between the samples from normal donors and samples from transplant recipients undergoing immunosuppressive therapy. Thus, this Example unexpectedly demonstrates that the method of the invention is suitable analysis of the amount of nuclear NFkB using a procedure whereby immune cells are activated in whole blood.

### EXAMPLE 3

The results presented in this Example were performed using a commercially available imaging cell flow cytometer (Amnis Corporation, Seattle, Wash.) as in Example 1. The materials and methods utilized to obtain the results presented in the Example are essentially as follows:

Protocol to measure the NF-κB and/or NFAT activation potential in immunophenotypically defined cell populations: Obtain peripheral blood sample in sodium heparin container. Keep PBL sample at ambient room temperature. Prepare sufficient number of 15 mL polypropylene tubes (10-9152N, Niagara Scientific, Lancaster, N.Y.) to accommodate the number of variables to be tested. One tube is needed for the unstimulated control, one for the unstimulated cell line control (HL60 30 or Jurkat) and two (1 for HL60 and 1 for patient sample) for each of the stimulation conditions to be tested (e.g. PMA/Ion, TNFα, CD3/CD28, etc.). Aliquot 500 μl whole blood per tube for the patient samples. For cell line controls, add enough volume from culture flask to yield 2×10⁶ cells/tube. Spin down the cell lines in the centrifuge to obtain a cell pellet (4 minutes@1800 rpm). Remove supernatant and agitate tubes to resuspend cells in residual volume and add 1 mL normal growth media (RPMI 1640) to cell pellets. Add the appropriate amount of stimulant to each tube and incubate for 30 minutes in a 37 degree incubator (or waterbath) for approximately 30 minutes. Place the whole blood samples on ice for immunophenotyping.

Cell line controls are washed in 1× Phosphate Buffered saline solution (1×PBS) (Mediatech, Herndon, Va.), and then fixed by adding 1 ml of a 4% solution of methanol free Formaldehyde (Polysciences, Warrington, Pa.) at room temperature for 10 minutes. Cells are washed twice in 1×PBS, spin, and resuspend cell pellets in residual volume (they are now ready to be probed for NF-κB and/or NFAT and are left at room temperature until whole blood samples are ready). Before adding antibody cocktail for cell surface immunophenotyping of the whole blood, block with normal mouse IgG (10400C, Invitrogen, Carlsbad, Calif.) for 10 minutes on ice. Without washing, add the antibody cocktail and incubate for 20 minutes on ice. After surface staining, blood samples need to be lysed and fixed using a commercially available Lyse/Fix reagent (558049, BD Biosciences, San Jose, Calif.). Add 9.5 mLs of a 1× solution of this reagent, pre-warmed to 37 degrees, to each whole blood sample, mix by gently inverting each tube several times, and incubate in a 37 degree waterbath for 10 minutes. Spin down for 4 minutes@1800 rpm and resuspend pellets in residual volume. Wash cells with 1×PBS, spin again, and resuspend pellets in residual volume. Cells are now ready to be probed for NF-κB and/or NFAT. Unconjugated anti-NF-κB-p65 antibody (sc-372, Santa Cruz Biotechnology, Santa Cruz, Calif.) is diluted 1:50 in permeabilization buffer (0.1% Triton-X-100 (648463, EMD Biosciences, San Diego, Calif.) in 1×PBS) and added to all whole blood and cell line controls and left at room temperature for 20 minutes. Alternatively, anti-NFAT antibodies (e.g. antibodies to NFAT1). Samples are washed with 1×PBS, spin, resuspended in residual volume and stained with the secondary FITC conjugated F(ab′)2 Fragment specific donkey anti rabbit IgG (711-096-152, Jackson Immunoresearch, Westgrove, Pa.) diluted 1:100 in permeabilization buffer, covered, and left at room temperature for 20 minutes. Samples are then washed in 1×PBS and are ready to be run on the imaging cell sorting machine. 60 μL volumes of each sample are transferred to 1.5 mL polypropylene tubes (T4816, Sigma-Aldrich, St. Louis, Mo.) and 10 μL of 5 μg/mL DAPI (D3571, Invitrogen, Carlsbad, Calif.) is added to each. Fluors are excited using a combination of relevant lasers, for example 405 nm (for DAPI), 488 nm (for FITC) and 658 nm (for APC). Cell classifiers are set on ‘Area’ to eliminate speed beads and cell clumps; Area Minimum=50, Area Maximum=250. A cell classifier is also set on Intensity of the DAPI signal to include only nucleated cells; Intensity Minimum=50.

At least 20,000 events are collected per whole blood sample, and 5000 for cell line controls. The aim is to collect at least 500 target cells. Single color controls are run for all fluors for compensation. 500 events are collected, with a cell classifier of Intensity Minimum set on the channel of interest. Brightfield and scatter laser are turned off for these samples.

Samples are analyzed using IDEAS® software (Amnis, Seattle, Wash.). Following compensation of the images, cell populations are hierarchically gated for single cells that are in focus, and are positive for both DAPI and NF-κB-p65 and/or 25 NFAT (e.g. NFAT1). After phenotyping gates are applied, the aim is to have acquired at least 500 cells of each targeted subpopulation. Following data acquisition, the spatial relationship between the NF-κB-p65 and the NFAT (e.g. NFAT1) and nuclear images is measured using the ‘Similarity’ feature in the IDEAS® software package. The ‘Similarity Score’ (SS), a log-transformed Pearson's correlation coefficient between the pixel values of two image pairs, provides a measure of the degree of nuclear localization of NF-κB-p65 and/or NFAT (e.g. NFAT1) by measuring the pixel intensity correlation between the anti-NF-κB/p65-FITC and/or anti-NFAT-FITC and DAPI images. The relative shift in this distribution between two populations (e.g., control versus stimulated cells) is calculated using the Fisher's Discriminant ratio (Rd value). Preparation of buffers applicable to the method: Permeabilization Buffer—500 μL 10% Triton-X-100; 24.5 mL 1×PBS; 4% Formaldehyde—20 mL 10% Formaldehyde; 30 mL 1×PBS; 1×BD Lyse/Fix Buffer—5× Lyse/Fix; Dilute to 1× in ddH₂O.

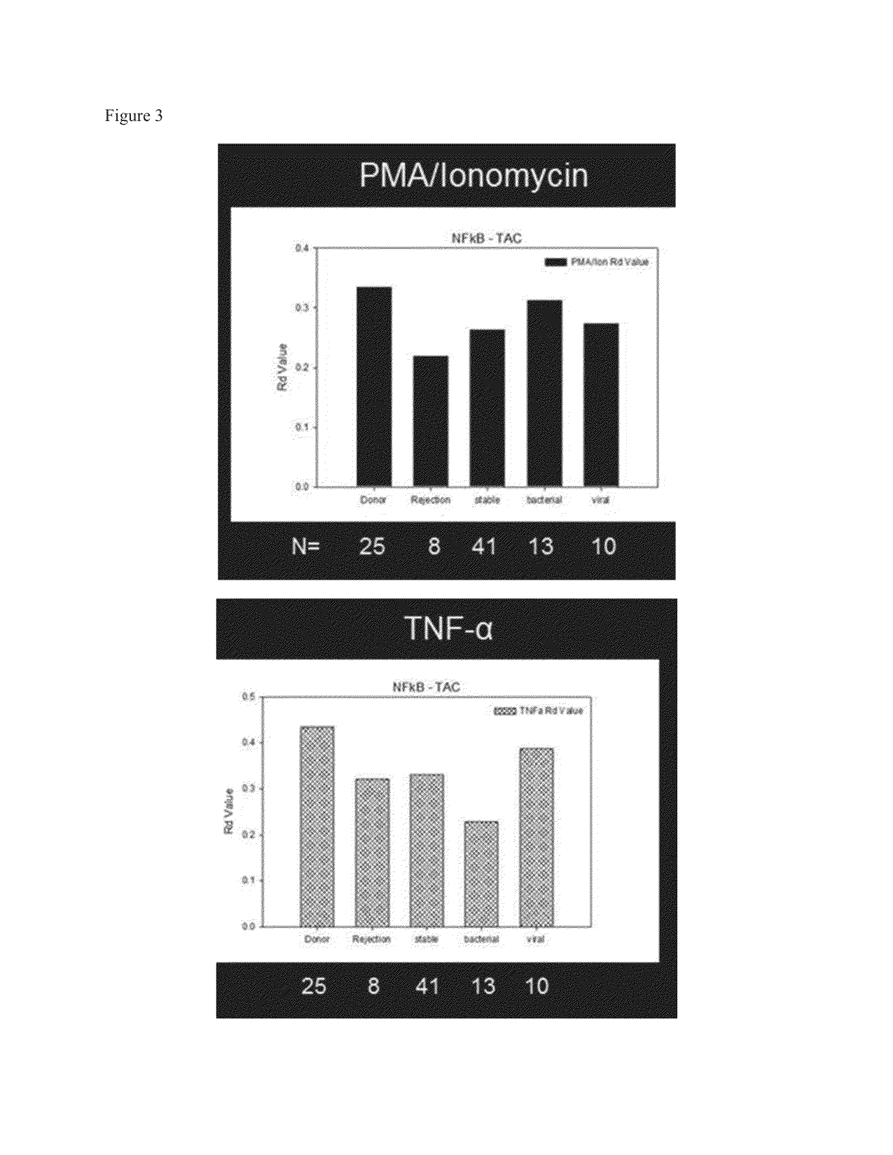

The foregoing materials and methods or minor modifications of them were used to obtain the results which are graphically represented in FIGS. 3-11.

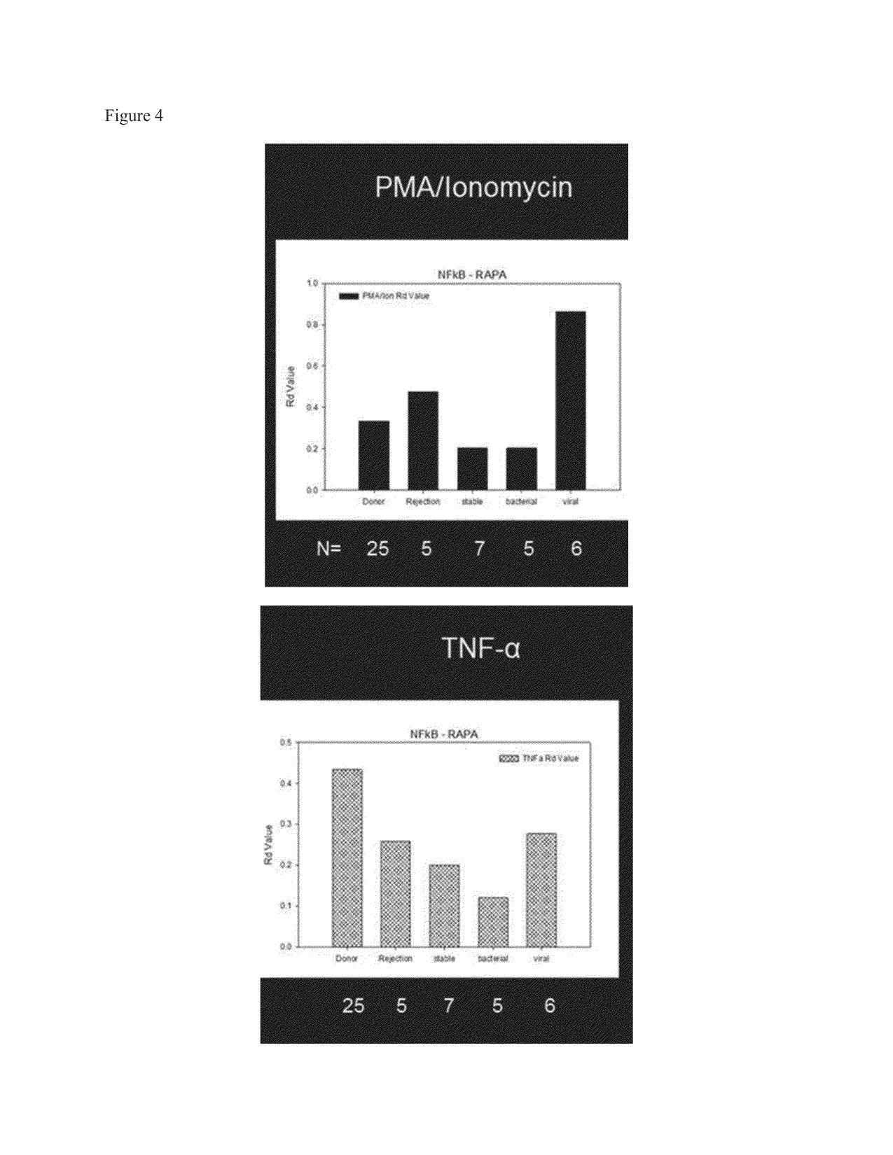

FIG. 3 shows the inhibition of PMA/Ion- or TNF-alpha-induced activation of NFkB for patients treated with a tacrolimus-based regimen categorized according to clinical presentation, while FIG. 4 shows the same relationship for patients treated with a rapamycin-based regimen. These data indicate TNF-alpha-induced NFkB activation is predictive for patients treated with a non-calcineurin inhibitor (i.e., rapamycin) regimen; in these experiments inhibition of PMA/Ion-induced activation is not was not predictive for either patient population.

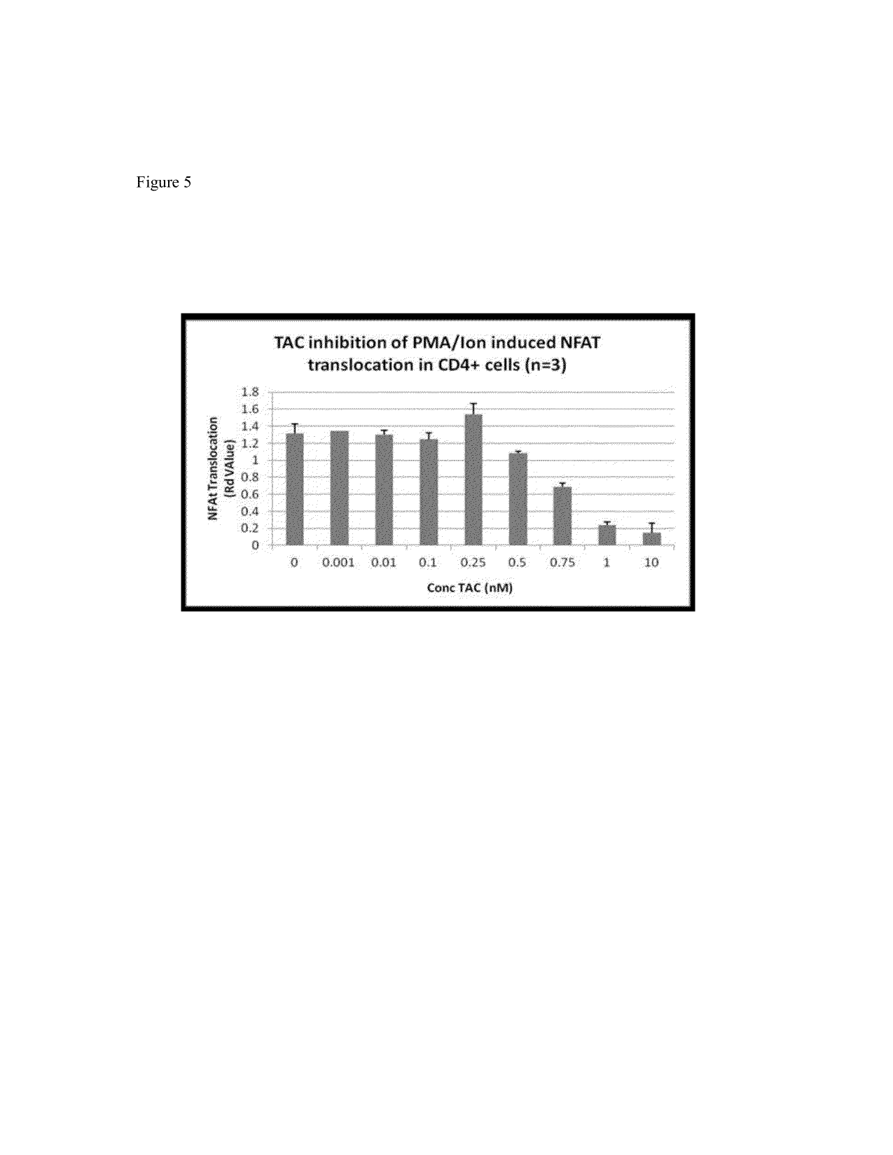

Since NFAT is a more direct target for calcineurin inhibitors this raised the question whether inhibition NFAT would be a preferable response parameter for calcineurin inhibitor (i.e., tacrolimus) based immunosuppressive regimen. Since the activation of NFAT is dependent on a cellular calcium-flux (for example as a consequence of T-cell receptor engagement) PMA/Ion was used as an activating source. The data in FIG. 5 demonstrate that in healthy donor peripheral blood, a dose-dependent inhibition of PMA/Ion-induced NFAT translocation is observed in the presence of increasing concentration of tacrolimus.

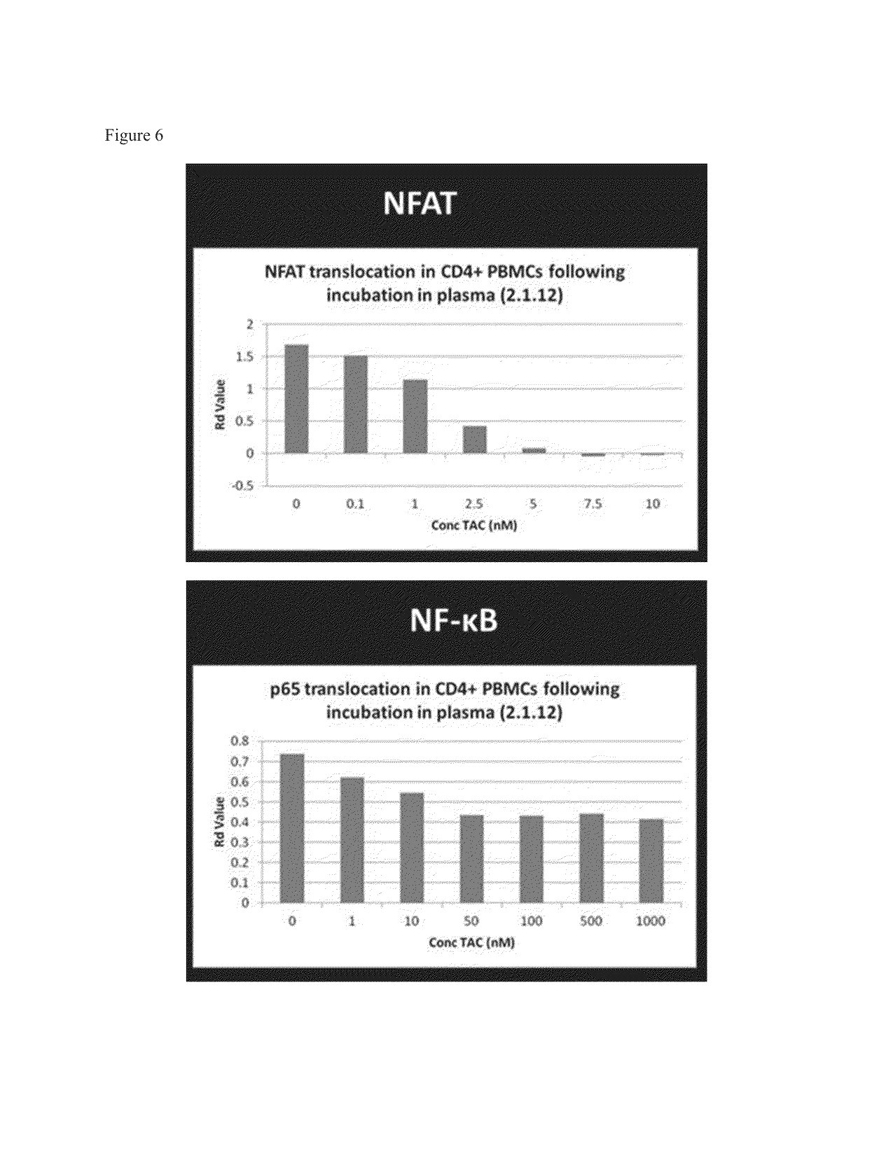

The data in FIG. 6 demonstrate a dose-dependent decrease of NFAT translocation potential in healthy donor CD4+ cells in the presence of increasing concentrations of Tacrolimus. This inhibitory effect is less pronounced for NFkB but can be seen at higher drug concentrations which are occasionally achieved in patients.

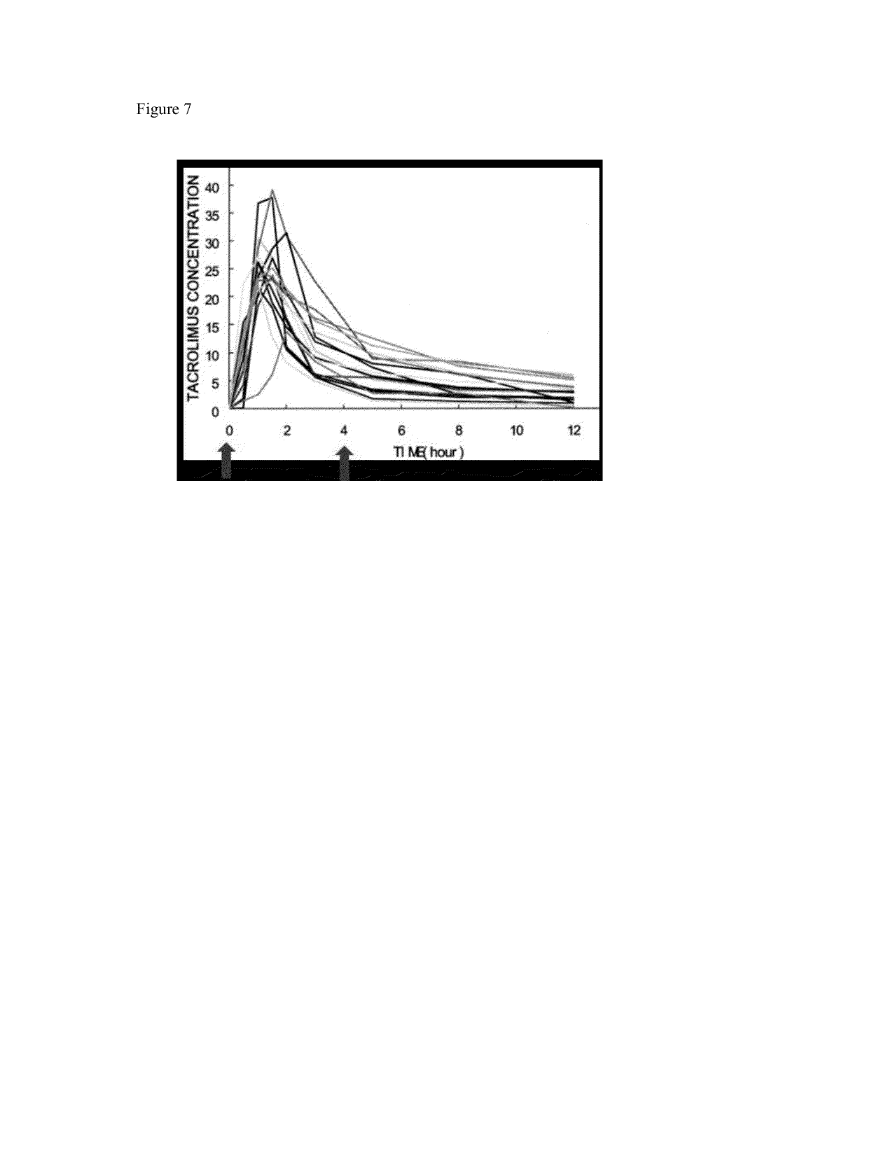

NFAT as a Tacrolimus target was tested in a cohort of stable transplant recipients by determining the PMA/Ion-induced activation potential before (0 h) and after (4 h) a dose of Tacrolimus with the rationale that the intracellular pharmacokinetics of tacrolimus would follow the plasma pharmacokinetics with a slight delay and that the expected different concentrations of tacrolimus should results in different levels of inhibition. FIG. 7 shows typical plasma pharmacokinetics of tacrolimus following a single oral dose (from Chen et al: Transplantation Proc 37(10:4246, 2005). We did not determine a significant difference between the NFAT activation potential in CD4+ or CD8+ cells between the 2 time points tested.

Since the same dose of tacrolimus can result in inter-patient differences in the drug's trough levels this correlation was also investigated. Neither for CD4+ nor CD8+ cells was a correlation found between trough tacrolimus levels achieved and the NFAT activation potential.

In order to determine if the observed lack of inhibition of NFAT activation could be due to the absence of bioavailable tacrolimus levels, the inhibitory effect of patient plasma obtained during tacrolimus treatment on cells naïve to previous tacrolimus exposure (Jurkat cell line) was evaluated.

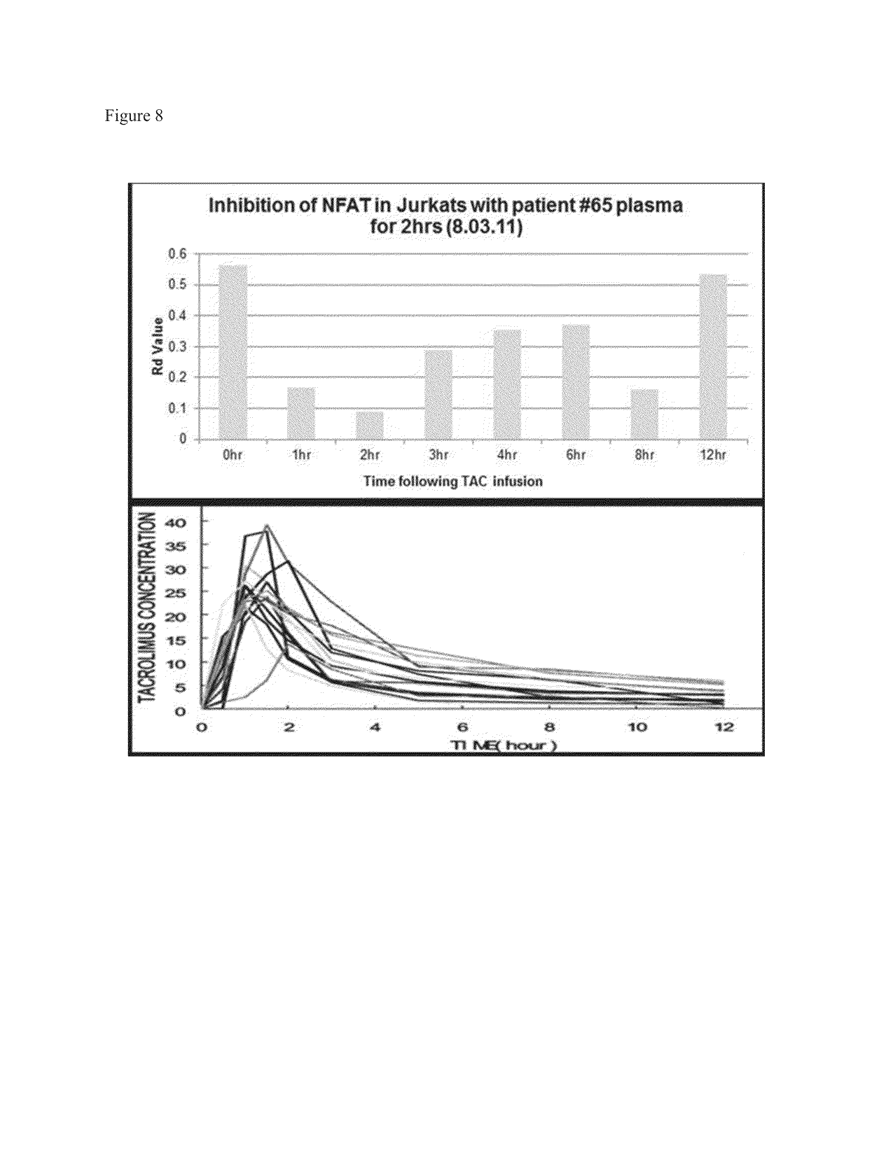

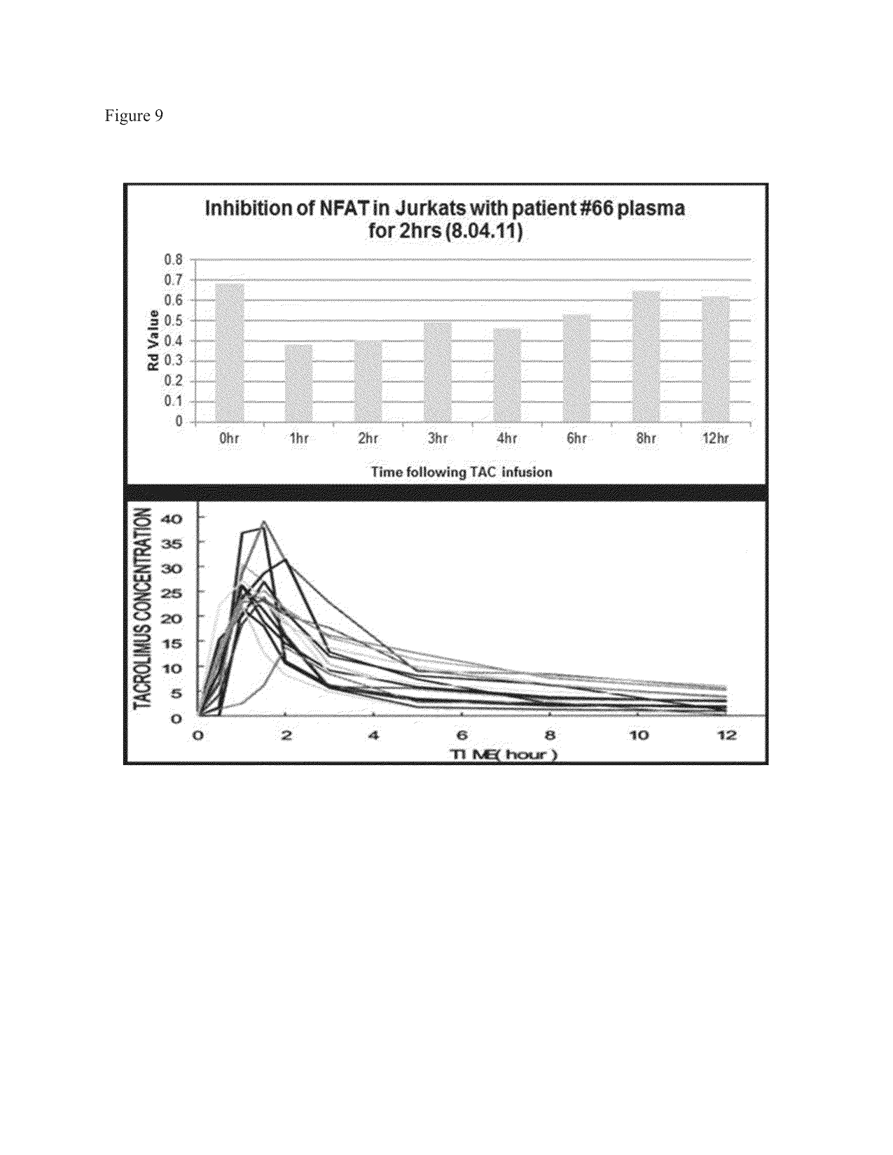

The data in FIGS. 8 and 9 demonstrate that the inhibitory effect of tacrolimus on NFAT in cell naïve to previous tacrolimus exposure closely mirrors that of the expected plasma levels. Therefore, the lack of predictive value of measuring the NFAT activation potential in CD4+ and CD8+ T cells may be due an intrinsic biological property in these autologous cells related to their chronic exposure to Tacrolimus.

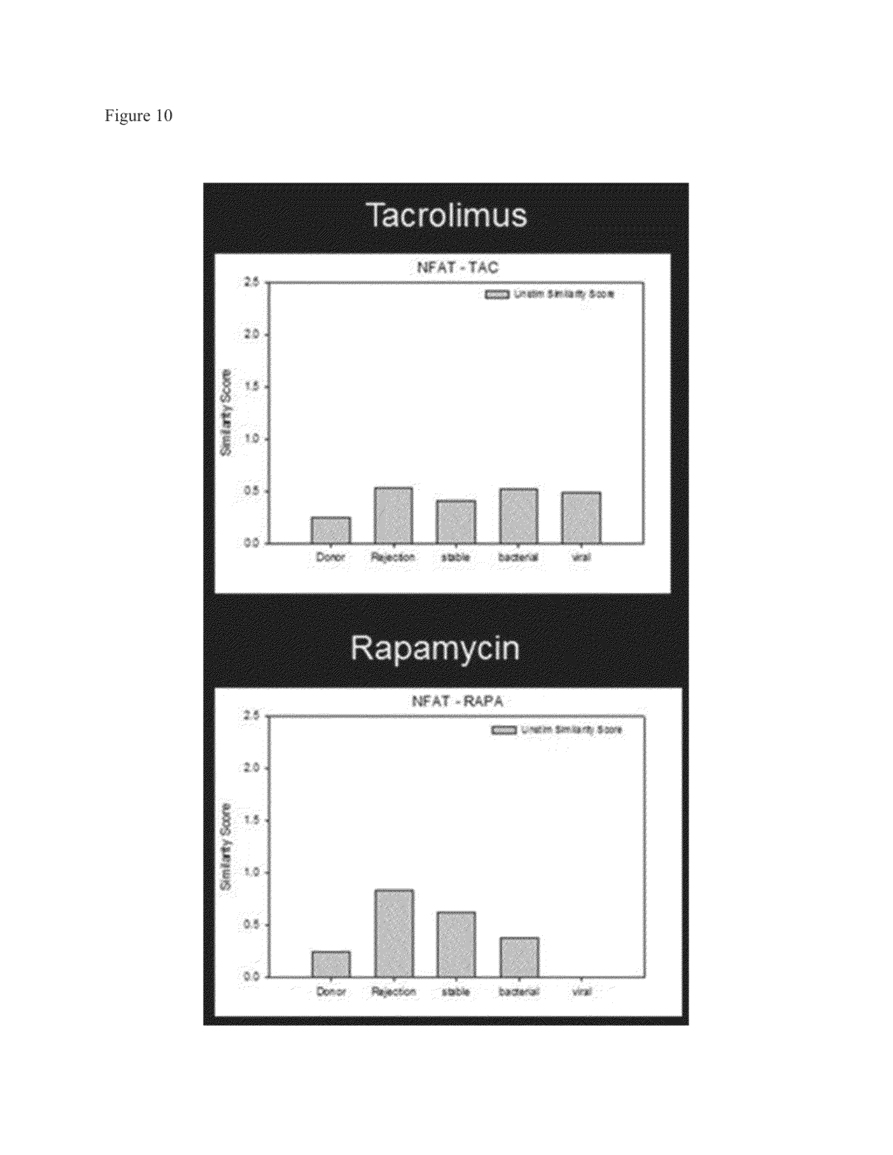

Of interest is the observation shown in FIG. 10 of the correlation between baseline NFAT activity levels and predictive value for clinical presentation. These data indicate that the baseline NFAT activation levels correspond with clinical presentation for patients treated with Rapamycin which is not directly targeting NFAT (n=6).

To summarize some of the foregoing results, the method of the invention was performed for a patient population who received two different immunosuppressive regimens. The most prevalent therapy was based on the calcineurin inhibitor tacrolimus and combined this with mycophenolic acid and prednisone (TMP). The second regimen was based on the the mTOR inhibitor Rapamycin (sirolimus) in combination with mycophenolic acid and prednisone (RMP). The data demonstrate that the NFAT and NFkB activation potential have predictive values which are related to the type of activation and immunosuppressive regimen used.

In the case of NFkB, inhibition of TNF-alpha-induced NFkB activation is predictive and this predictive value is stronger for patients treated with a RMP regimen.

NFAT is a more direct target for calcineurin inhibitors. Since the activation of NFAT is dependent on a cellular calcium-flux (for example as a consequence of T-cell receptor engagement) PMA/Ion was used as an activating source. In vitro, a strong correlation is observed between extracellular concentrations of the calcineurin inhibitor tacrolimus and the PMA/Ionomycin-induced NFAT1 activation potential quantified by the commercially available ImageStream platform. This dose-response relationship is observed when tacrolimus is spiked into tissue culture media, plasma or whole blood as the extracellular environment as well as when variable tacrolimus plasma concentrations are present following oral administration. The dose-response relationship is observed in cells that are naïve to prior tacrolimus exposure (Jurkat cell line or healthy donor CD4+ or CD8+ T cells). In T cells from transplant recipients who have been chronically exposed to tacrolimus, no statistically significant dose-response relationship was observed. In order to determine if the observed lack of statistically significant inhibition of NFAT1 activation in the patients' T cells was possibly due to the absence of bioavailable tacrolimus levels in vivo, the inhibitory effect of patient plasma obtained during tacrolimus treatment on cells naïve to previous tacrolimus exposure (Jurkat cell line) was evaluated and a good dose-response was observed. Therefore, the lack of statistically significant predictive value of measuring the NFAT1 activation potential in patient CD4+ and CD8+ T cells is likely due an intrinsic biological property in these ‘autologous’ cells related to their chronic exposure to Tacrolimus. Based on these data to date we hypothesize that in patients treated with a TMP-immunosuppressive regimen in which NFAT is a primary target, the NFkB pathway serves a compensatory role and therefore measuring its baseline and activation potential is expected to be predictive for the level of clinical immunosuppression achieved. Vice versa, in patients treated with an RMP-immunosuppressive regimen in which NFAT is not a primary target, the NFAT pathway serves a compensatory role and therefore measuring its baseline and activation potential will be predictive for the level of clinical immunosuppression achieved.

In view of the foregoing, the invention in certain embodiments comprises determining multivariate parameters of response that take into consideration both the NFAT and NFkB pathways, base-line as well as activation potential, which facilitates altering treatment regimens according to drugs used (calcineurin vs non-calcineurin inhibitors).

The strong dose-response correlation that has been observed between extracellular concentrations of tacrolimus and inhibition of NFAT1 activation potential in T cells naïve to prior tacrolimus exposure suggest that this measurement can be used as an ex-vivo assessment of sensitivity of the drug's target (NFAT) in patient CD4+ and CD8+ T cells and thus to individually optimize dose prior to transplant. In order to correlate with clinical immunosuppression levels post-transplant, the activity of compensatory immunoregulatory pathways such as NFkB can also be taken into consideration.

While the invention has been described through illustrative examples, routine modifications will be apparent to those skilled in the art, which modifications are intended to be within the scope of the invention.

We claim:

1. A method for determining immunosuppression in an individual comprising:
i) obtaining a biological sample comprising nucleated blood cells from the individual;
ii) contacting the cells in the sample with an activating agent to obtain activated cells;
iii) testing the cells to determine an amount of nuclear NFkB and/or NFAT in the activated cells; and
iv) comparing the amount of nuclear NFkB and/or NFAT in the activated cells to a control;
wherein more nuclear NFkB and/or NFAT relative to the control is indicative of insufficient immunosuppression in the individual;
wherein less nuclear NFkB and/or NFAT relative to the control is indicative of excessive immunosuppression in the individual; and
wherein the same amount of nuclear NFkB and/or NFAT as the control is indicative of an appropriate amount of immunosuppression in the individual.

2. The method of claim 1, wherein:
i) a second sample comprising nucleated blood cells is obtained from the individual, wherein the second sample is not contacted with the activating agent, wherein a second amount of nuclear NFkB is determined from cells in the second sample to obtain a non-activated amount of nuclear NFkB, and wherein the non-activated amount of nuclear NFkB is compared to the amount of nuclear NFκB in the activated cells of claim 1; or
ii) a second sample comprising nucleated blood cells is obtained from the individual, wherein the second sample is not contacted with the activating agent, wherein a second amount of nuclear NFAT is determined from cells in the second sample to obtain a non-activated amount of nuclear NFAT, and wherein the non-activated amount of nuclear NFAT is compared to the amount of nuclear NFAT in the activated cells of claim 1; or
iii) steps i) and ii) are both performed.

3. The method of claim 1, wherein the sample comprising nucleated blood cells is whole blood.

4. The method of claim 1, wherein the activated cells are selected from the group consisting of T cells, B cells, monocytes, polymorphonuclear leukocytes, eosinophils, and combinations thereof.

5. The method of claim 1, wherein the activated cells are CD3+ cells, CD4+ cells, CD8+, CD20+ cells, or a combination thereof.

6. The method of claim 1, wherein the determining the amount of nuclear NFκB in the activated cells is performed using a detectably labeled antibody directed to a p65 subunit of the NFκB.

7. The method of claim 1, wherein the activating agent is selected from phorbol 12-myristate 13-acetate (PMA) with ionomycin (ion), tumor necrosis factor alpha (TNF-alpha), and anti-CD3/CD28 antibodies.

8. The method of claim 7, wherein the amount of nuclear NFkB is determined, wherein the activating agent is TNF-alpha, and wherein the individual is undergoing immunosuppression therapy with a non-calcineurin inhibitor.

9. The method of claim 8, wherein the non-calcineurin inhibitor is rapamycin.

10. The method of claim 1, wherein the determining the amount of nuclear NF B and/or the amount of nuclear NFAT in the activated cells is performed using an imaging cell sorting machine.

11. The method of claim 10, wherein the amount of nuclear NFκB is determined.

12. The method of claim 1, wherein the individual is a recipient of an organ transplantation.

13. The method of claim 1, further comprising communicating to a health care provider a determination that the amount of nuclear NFkB and/or NFAT is indicative of insufficient, excessive or appropriate immunosuppression in the individual.

14. The method of claim 1, further comprising modifying immunosupprssion dosing for the individual subsequent to determining the amount of nuclear NFkB and/or NFAT is indicative of insufficient or excessive immunosuppression.

15. The method of claim 1, wherein the individual is a human being.

16. A method for determining nuclear translocation of NFkB and/or NFAT for an individual undergoing immunosuppression, the method comprising:
i) obtaining a biological sample comprising nucleated blood cells from the individual;
ii) contacting the cells in the sample with an activating agent to obtain activated cells;
iii) testing the cells to determine an amount of nuclear NFκB and/or NFAT in the activated cells.

17. The method of claim 16, wherein the individual is a recipient of an organ transplantation, or is a candidate to receive an organ transplantation.

18. The method of claim 16, wherein the nucleated cells are T cells.
